(19) United States
(12) Patent Application Publication
(10) Pub. No.: US 2003/0219791 A1
(43) Pub. Date: Nov. 27, 2003

(54) Transducer of MAS signalling

(76) Inventors: Vibeke Westphal Stennicke, Kokkedal (DK); Peder Lisby Norby, Kobenhavn 0 (DK); Christian Grondahl, Vaerlose (DK); Philip Wahl, Hillerod (DK)

Correspondence Address: Reza Green, Esq., Novo Nordisk Pharmaceuticals, Inc., 100 College Road West, Princeton, NJ, 08540, US

(21) Appl. No.: 10/370,860

(22) Filed: Feb. 20, 2003

(30) Foreign Application Priority Data
Feb. 22, 2002 (DK) PA 2002 00277

(51) Int. Cl. C12Q 1/68; G01N 33/53; G01N 33/567; C07H 21/04; C12N 9/64; C12N 1/21; C12N 1/18; C12N 5/06; C12P 21/02

(52) U.S. Cl. 435/6; 435/7.2; 435/69.1; 435/226; 435/320.1; 435/325; 435/252.3; 435/254.2; 536/23.2

(57) ABSTRACT

Two receptors of meiosis activating sterols designated SAM1a and SAM1b have been identified.

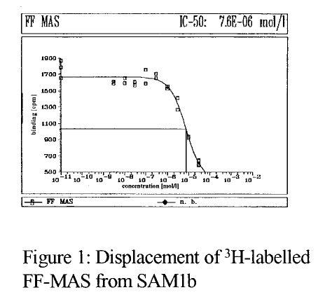

### CROSS-REFERENCE TO RELATED APPLICATIONS

This application claims priority under 35 U.S.C. 119 of Danish application nos. PA 2000 01259 filed Aug. 25, 2000 and PA 2002 00277 filed Feb. 22, 2002 and the benefit of application no. PCT/DK01/00550 filed Aug. 20, 2001, under 35 U.S.C. 120, the contents of which are fully incorporated herein by reference.

### FIELD OF THIS INVENTION

The present invention relates to signal transducers of FF-MAS, polynucleotides coding for signalling proteins of FF-MAS, probes hybridising with nucleic acids encoding signalling proteins of FF-MAS, DNA constructs comprising a sequence encoding signalling proteins of FF-MAS, culture cell lines wherein the DNA sequence encodes signalling proteins of FF-MAS, antibodies specifically binding to signalling proteins of FF-MAS, hybridoma producing monoclonal antibodies specifically binding to signalling proteins of FF-MAS, and methods for detecting the presence of a compound having affinity to signalling proteins of FF-MAS.

### BACKGROUND OF THIS INVENTION

Since the first IVF pregnancy was delivered in 1978, this procedure has resulted in more than a million of pregnancies and opened a vast new frontier of research and treatment for the infertile couples. Still, there is a significant need for improved infertility treatment modalities today. It is presumed that more than one out of seven couples experience problems with sub fertility or infertility.

IVF of human oocytes has become commonly used for the treatment of female and male sub fertility. The standard IVF treatment includes a hormone stimulation of the female patient. The aspirated oocyte is subsequently fertilised in vitro and cultured. Continuous efforts have been made to optimise and simplify this procedure. Nevertheless, the overall pregnancy rate has not be increased significantly over about 20% with the current treatment modalities. In a large European survey of IVF patients, it was found that 7.2 oocytes out of 11.5 aspirated oocytes per patient had undergone resumption of meiosis immediately before fertilisation, only 4.3 oocytes were fertilised and only 2.2 oocytes reached the 8-cell embryo stage after fertilisation and in vitro culture (ESHRE, Edinburgh, 1997).

Due to the very unpredictable quality of the state of the art embryos today, usually more than one embryo is being transferred to give a reasonable chance of success. Therefore, it is common to transfer 2-3 embryos (up to 5 embryos in some countries), which carries the very large side effect of multiple pregnancies with great discomfort and risk to both patient and children. Moreover, it has been estimated that the increased health care expenses due to multiple birth (twins, triplets etc.) is exceeding the entire IVF expenses.

Hence, there are several disadvantages with the current treatment.

Furthermore, weight gain, bloating, nausea, vomiting, labile mood and other patient discomforts together with patient reluctance to inject themselves are reported as disadvantages.

It is known from WO 96/00235 that certain sterol derivatives can be used for regulating meiosis. An example of such a sterol is 4,4-dimethyl-5α-cholesta-8,14,24-triene-3β-ol (hereinafter designated FF-MAS).

Hence, at present, in vitro maturation in humans has proven highly unsuccessful despite substantial interest and clinical efforts. Similarly with other mammals.

One way of trying to find compounds which effectively regulate meiosis is the use of pertinent receptors.

Receptors are defined as proteinaceous macromolecules that perform a signal transducing function upon ligand binding.

Signal tranducers (transducers of MAS-signalling) are defined as protein molecules that are directly or indirectly stimulated by a ligand/receptor interaction and transduce this signal further to other molecules and eventually leads to a change in enzymatic activity or functional state of effector molecules e.g. higher gene transcription, activation of inactive proteins or GVB in oocytes. Hence, signal transducers do not need to interact physically with the stimulating molecule. Signal transducers include, but are not limited to, kinases, phosphatases and proteases. Signal transducers are often present in the cytoplasmic compartment of the cell, but may also be found in the nucleus, attached to the plasma membrane, or associated with intracellular organelles.

The present invention does not relate to MAS receptors or to MAS-signalling proteins (transducers of MAS-signalling) which bind directly to FF-MAS as the ligand. The pharmaceutical industry in recent years has oriented its research to focus on the role of signal transducers in disease or injury and to design drugs, generally low molecular weight substances, that are capable of binding to the signal transducer. Drugs identified in this initial screen are then tested for the activity in vivo or in tissue explants. As a result, conventional techniques do not lend themselves to large-scale screening. Tissue samples or isolated cells containing the target signal transducer, for example ovarian tissue, are costly to obtain, present in limited quantity, and difficult to maintain in a functionally viable state. Additionally, it is often difficult to reliably and reproducibly administer the candidate drug to tissue samples. Screening assays using primary explants in tissue culture are undertaken in larger scale than is possible with tissue samples. However, it is more difficult to assay physiological effect and the assays are subject to interference from many sources, for example culture media or cultivation conditions. Finally, assays using signalling protein isolated from natural materials have the disadvantage that the signalling protein is subject to natural variability and suitable natural sources may not always be available. It is an object herein to provide readily reproducible, simple assay systems that can be practiced on a large scale for determining not only ligand binding but also the character of the binding as agonistic or antagonistic.

Similarly, meaningful clinical diagnosis often depends upon the assay of biologically active ligand without interference from inactive forms of the ligand, for example, ligands that have been subject to enzymatic or other processes in the test subject that change or even eliminate the activity of the ligand. Immunoassay methods are widely used in determining ligands in test samples. However, it is often quite difficult to identify antibodies that are able to discriminate between the active and inactive forms of a ligand. Signalling transducers have frequently been used in place of antibodies as analyte binding reagents. However, not all substances that bind to signal transducers are necessarily capable of inducing signal transduction, i.e., active biologically. It is an object herein to provide a method that will identify ligands in clinical test samples which are active in inducing or inhibiting signal transduction.

Cytoplasmic proteins can act as signalling molecules in cascading the stimulus from the ligand to cellular events. Various signalling protein types make use of different path ways (for example small G proteins, calcium fluxes, phospatases, and lipases), all of them resulting in changes of enzymatic activity or gene transcription. Meiotic activating sterols (hereinafter designated MAS) constitute active signalling molecules first identified in follicular fluid and in bull testicular tissue. The sterols are described by Byskov 1995 and Grøndahl et al. (Biol. Reprod. 58 (1998), 1297 et seq.) and in WO 96/00235, 96/27658, 97/00884, 98/28323, 98/54965 and 98/55498, more specifically in claim 1 thereof, as being potent activators of the meiotic process. No receptors or signalling proteins have been described to directly or indirectly signalling the meiotic effect of MAS sterols. Before this invention, the presence of the nature of a putative MAS receptor protein or a signalling protein has not previously been identified, although its presence has been suggested, for example, by Grøndahl et al. (Biol. Reprod. 62 (2000), 775 et seq.).

On Sep. 29, 2000, the nucleotide and amino acid sequence of clone NT2RM2001632 was released with accession number AK022554 and on May 10, 2001, the nucleotide and amino acid sequence of clone NT2RP2000448 was submitted with accession number AK027535. No utility or action was mentioned for these clones.

There remains considerable need for an isolated and purified transducer of MAS-signalling, as well as systems capable of expressing a transducer of MAS-signalling separate from other signal transducers. Further, it would be desirable to specifically identify the presence of a transducer of MAS-signalling in cells and tissues, thereby avoiding the time-consuming, complex and non-specific functional pharmacological assays. It would also be desirable to screen and develop new agonists and/or antagonists specific for a transducer of MAS-signalling for the use of antiinfertility or contraception drugs, but to date this has not been possible. Quite surprisingly, the present invention fulfils these and other related needs.

### SUMMARY OF THE INVENTION

Now, the present invention provides the nucleotide sequence of a signal transducer of meiotic acting sterols (MAS). The present invention provides isolated and substantially pure transducers of MAS-signalling and fragments thereof. These signalling proteins have been shown to be involved in the gamete maturation process induced by 3β-hydroxy-4,4-dimethylcholest-8,14,24-triene (hereinafter designated FF-MAS), specifically inducing germinal vesicle breakdown (hereinafter designated GVB) in mouse oocyte cultured in vitro. Hence, a transducer of MAS-signalling is defined as a proteinaceous macromolecule that perform a signal transducing function stimulated by FF-MAS.

A transducer of MAS-signalling is any protein related to the protein SAM1a or SAM1b that possess the same functional characteristic upon stimulation with FF-MAS or other endogenous meiosis activating sterols, for example, 3β-hydroxycholest-8,14-diene; 3β-hydroxy-4,4-dimethylcholest-8,24-diene; and 3β-hydroxycholest-8,24-diene, or their metabolites (as ligand). Functional characteristics include binding, receptor activation, phosphorylation, and subsequent germinal vesicles breakdown (GVB) in oocytes. The amino acid sequence of SAM1a and SAM1b is stated in SEQ ID NO: 2 and SEQ ID NO: 4, below.

Having provided such transducer of MAS-signalling in isolated or purified form, the invention also provides antibodies to the transducer of MAS-signalling, in the form of antisera and/or monoclonal antibodies.

In another aspect, the invention provides the ability to produce the transducer of MAS-signalling and polypeptides or fragments thereof by recombinant means. The expressed transducer of MAS-signalling or fragments may or may not have the biological activity of native signalling protein. Accordingly, isolated and purified polynucleotides are described which code for the signalling protein and fragment thereof, where the polynucleotides may be in the form of DNA, such as cDNA, or RNA. Based on these sequences, probes may be used to hybridise and identify these and related genes which encode transducers of MAS-signalling. The probes may be full length cDNA or as small as form 14 to 25 nucleotide, more often though from about 40 to about 50 or more nucleotides.

In related embodiments, the invention concerns DNA constructs which comprise a transcriptional promoter, a DNA sequence which encodes the signalling protein or fragment, and a transcriptional terminator, each operably linked for expression of the transducer of MAS-signalling. For expression, the construct may also contain at least one signal sequence. Further, for large-scale production, the expressed transducer of MAS-signalling may also be isolated from the cells by, for example, immunoaffinity purification.

Cells or bacteria which express the transducers of MAS-signalling may also be used to identify compounds which can alter the signalling protein-mediated metabolism of a cell. Compounds may be screened for binding to the transducer of MAS-signalling, and/or for effecting a change in transducer of MAS-signalling-mediated metabolism in the host cell. Agonists and/or antagonists of the transducers of MAS-signalling may also be screened in cell-free systems using purified transducers of MAS-signalling or binding fragments thereof for the effect on ligand/transducer of MAS-signalling interaction, or using reconstituted systems such as micelles which also provide the ability to assess metabolic changes.

In yet other embodiments, the invention relates to methods for diagnosis, where the presence of a mammalian transducer of MAS-signalling in a biological sample may be determined. For example, a monospecific antibody which specifically binds the transducer of MAS-signalling is incubated with the sample under conditions conducive to immune complex formation, which complexes are then detected, typically by means of a label such as an enzyme, fluorophore, radionuclide, chemiluminiscer, particle, or a second labelled antibody. Thus, means are provided for immunohistochemical staining of tissues, including ovarian or testicular tissues, for the subject transducer of MAS-signalling.

Based upon the similarity in sequence and the shared presence of a sterol binding domain at the protein level, the transducer of MAS-signalling of this invention can be said to belong to a novel super family of oxysterol binding proteins (hereinafter designated OSBP) recently published in J.Lipid.Res. 40 (1999), 2204. No function whatsoever in gamete maturation of either gender or regulation of any meiotic processes has been assigned to this OSBP family.

### BRIEF DESCRIPTION OF THE FIGURES

SEQ ID NO: 1 and SEQ ID NO: 3 are the nucleotides of the cDNA from two mouse MAS signalling peptides, designated SAM1a and SAM1b, respectively, and having the amino acid sequences stated in SEQ ID NO: 2 and SEQ ID NO: 4, respectively. SEQ ID NO: 5 and SEQ ID NO: 7 are the nucleotides of the cDNA from two human MAS signalling peptides, designated SAM1a and SAM1b, respectively, and having the amino acid sequences stated in SEQ ID NO: 6 and SEQ ID NO: 8, respectively. SEQ ID NO: 9 through 14 are the nucleotides referred to in example 2.

### DESCRIPTION OF THE PREFERRED EMBODIMENTS

The present invention presents the means to identify agonists and antagonists of the transducer of MAS-signalling function, said mean being isolated transducer of MAS-signalling. The term “transducer of MAS-signalling” refers to any proteins either derived from a naturally occurring transducer of MAS-signalling, or which shares significant structural and functional characteristics peculiar to a naturally occurring transducer of MAS-signalling. Such a transducer of MAS-signalling may result when regions of a naturally occurring transducer of MAS-signalling are deleted or replaced in such a manner as to yield a protein having a similar function. Homologous sequences, allelic variations, and natural mutants; induced point, deletion, and insertion mutants; alternatively expressed variants; proteins encoded by DNA which hybridise under high or low stringency conditions to nucleic acids which encode naturally occurring transducer of MAS-signalling; proteins retrieved from naturally occurring materials; and closely related proteins retrieved by antisera directed against transducer of MAS-signalling are also included.

By transducer of MAS-signalling “ligand” is meant a molecule capable of being bound by the transducer of MAS-signalling, a transducer of MAS-signalling analogue, or chimeric transducer of MAS-signalling similarly as described in U.S. Pat. No. 4,859,609, incorporated by reference herein. The molecule may be chemically synthesised or may occur in nature. Ligands may be grouped into agonists and antagonists. Agonists are those molecules whose binding to a protein induces the response pathway within a cell. Antagonists are those molecules whose binding to a protein blocks the response pathway within a cell.

By “isolated” transducer of MAS-signalling is meant to refer to transducer of MAS-signalling which is in other than its native environment such as a mammalian oocyte, including, for example, substantially pure transducer of MAS-signalling as defined herein below. More generally, isolated is meant to include transducer of MAS-signalling as a heterologous component of a cell or other system. For example, transducer of MAS-signalling may be expressed by a cell transfected with a DNA construct which encodes transducer of MAS-signalling, separated from the cell and added to micelles which contain other selected signalling proteins.

By purified transducer of MAS-signalling is meant transducer of MAS-signalling having a purity of at least 50%, preferably at least 80%, more preferred at least 90% (w/w). Human SAM1a and SAM1b are the clones NT2RP2000448 and NT2RM2001632, respectively.

By the term “high stringency” conditions is meant conditions under which the labeled probe, i.e., an oligonucleotide or polynucleotide of 25 or more contiguous nucleotides of SEQ ID NO: 1 or SEQ ID NO: 3 will hybridize with high specificity to the polynucleotide sequences to be tested containing few, preferably less than 10%, more preferred less than 5%, if any, mismatched bases. High-stringency hybridization conditions are described in, for example, Sambrook et al. 1989, “Molecular Cloning”, Cold Spring Harbor Laboratory Press.

In the case of probes in the form of polynucleotides of 200 bases or more, high stringency hybridization is achieved by incubating the probe and the membrane containing target DNA or mRNA in a buffer containing 6×SSC, 10% Dextran sulphate, 1% SDS, 5× Denhardts, 50 μg/ml salmon DNA (Stratagene), and 2×10⁶ cpm/ml of the radiolabeled probe. The incubation is at 68° C. with shaking or rotation for at least 2 hours, typically overnight. The membrane is then washed in 2×SSC, 0.1% SDS at 42° C. for 30 minutes, followed by a wash in 2×SSC, 0.1% SDS at 68° C. for 30 minutes, then a wash in 0.2×SSC, 0.1% SDS at 68° C., and finally a wash in 0.1×SSC, 0.1% SDS at 68° C. for 30 minutes. The membrane is exposed to x-ray film.

In case of oligonucleotide or polynucleotide probes 25-200 bases in length, high stringency hybridization is carried out in a solution containing 6×SSC, 0.05 M sodium phosphate (pH 6,8), 1 mM EDTA (pH 8,0), 5× Denhardts solution, 100 μg/ml salmon sperm DNA, 100 mg/ml dextran sulfate, and 180 pM of radiolabeled oligonucleotide (5×10⁵ to 1.5×10⁶ cpm/pmole). The hybridization temperature varies depending on the length of the probe. Sambrook et al. (supra, chapter 11) have described how to calculate, and experimentally verify, the conditions that will result in high-stringency hybridization. Hybridization is performed at 5-10° C. less than the Tₘ, and post-hybridization washes at 5° C. below the Tₘ, with the Tₘ calculated as

Tₘ=81.5−16.6(Log₁₀[Na+])+0.41(%G+C)−(600/N), where N=chain length.

Hybridization is done overnight with shaking or rotation. The membrane is then washed twice with 2×SSPE, 0.1% SDS at room temperature for 15 minutes, then with 0.2×SSPE, 0.1% SDS 5° C. below the Tₘ of the probe, for 60 minutes. The membrane is exposed to x-ray film.

It is obvious for the skilled art worker to test whether a specific protein is a transducer of MAS-signalling or, in other words, whether a specific protein is an analogue of SAM1a as specified in the claims below. In the present context, the term “analogue” is intended to indicate a naturally occurring variant (including one expressed in other animal species, for example, human, monkey, mouse or rat) of the transducer of MAS-signalling or a “derivative”, i.e., a polypeptide which is derived from the native transducer of MAS-signalling by suitably modifying the DNA sequence coding for the variant, resulting in the addition of one or more amino acids at either or both the C- and N-terminal ends of the native amino acid sequence, substitution of one or more amino acids at one or more sites in the native amino acid sequence, deletion of one or more amino acids at either or both ends of the native sequence or at one or more sites within the native sequence, or insertion of one or more amino acids in the native sequence.

In another aspect, the invention provides means for regulating the transducer of MAS-signalling/ligand function, and thus treating, therapeutically and/or prophylactically, a disorder which can be linked directly or indirectly to transducer of MAS-signalling or to its ligands, such as FF-MAS. By virtue of having the transducer of MAS-signalling of the invention, agonists or antagonists may be identified which stimulate or inhibit, respectively, the function of the transducer of MAS-signalling. With either agonists or antagonists, the metabolism and reactivity of cells which express the signalling protein are controlled, thereby providing a means to control meiosis in order to treat infertility or to achieve a novel principle of contraception.

Thus, the invention provides screening procedures for identifying agonists or antagonists of events mediated by the ligand/transducer of MAS-signalling function. Such screening assays may employ a wide variety of formats, depending to some extent on which aspect of the ligand or transducer of MAS-signalling interaction is targeted. For example, such assays may be designed to identify compounds which bind to the transducer of MAS-signalling and thereby block or inhibit function of the transducer of MAS-signalling. Other assays can be designed to identify compounds which can stimulate the transducer of MAS-signalling-mediated intracellular pathways. Yet other assays can be used to identify compounds which inhibit or facilitate the cellular response to the transducer of MAS-signalling.

In one functional screening assay, the initiation of fertilisation activation events are monitored in eggs which have been injected with, for example, mRNA which codes for transducer of MAS-signalling and subsequently exposed to selected compounds which are being screened, in conjunction with or apart form an appropriate ligand. See generally, Kline et al., Science 241 (1988), 464-467, incorporated herein by reference.

The screening procedure can be used to identify reagents such as antibodies which specifically bind to the transducer of MAS-signalling and substantially affect its function, for example. The antibodies may be monoclonal or polyclonal, in the form of antiserum or monospecific antibodies, such as purified antiserum or monoclonal antibodies or mixtures thereof. For administration to humans, for example, as a component of a composition for in vivo diagnosis or imaging, the antibodies are preferably substantially human to minimise immunogenicity and are in substantially pure form. By substantially human is meant generally containing at least about 70% human antibody sequence, preferably at least about 80% human, and most preferably at least about 90-95% or more of a human antibody sequence to minimise immunogenicity in humans.

Antibodies which bind to a transducer of MAS-signalling may be produced by a variety of means. The production of non-human antisera or monoclonal antibodies, for example, murine, lagomorpha equine, etc. is well known and may be accomplished by, for example, immunising the animal with the transducer of MAS-signalling molecule or a preparation containing a desired portion of the transducer of MAS-signalling molecule, such as that domain or domains which contributes to regulation. For the production of monoclonal antibodies, antibody-producing cells obtained from immunised animals are immortalised and screened, or screened first for the production of antibody which binds to the signalling protein and then immortalised. As the generation of human monoclonal antibodies to human transducer of MAS-signalling antigen may be difficult with conventional techniques, it may be desirable to transfer antigen binding regions of the non-human antibodies, for example, the F(ab′)₂ or hypervariable regions, to human constant regions (Fc) or framework regions by recombinant DNA techniques to produce substantially human molecules. Such methods are generally known in the art and are described in, for example, U.S. Pat. No. 4,816,397, and EP publications 173,494 and 239,400, which are incorporated herein by reference. Alternatively, one may isolate DNA sequences which code for a human monoclonal antibody or portions thereof that specifically bind to the human signalling protein by screening a DNA library from human B cells according to the general protocol outlined by Huse et al., Science 246 (1989), 1275-1281, incorporated herein by reference, and then cloning and amplifying the sequences which encode the antibody (or binding fragment) of the desired specificity.

In other embodiments, the invention provides screening assays conducted in vitro with cells which express the transducer of MAS-signalling. For example, the DNA which encodes the transducer of MAS-signalling or selected portions thereof may be transfected into an established cell line, for example, a mammalian cell line such as BHK and CHO, using procedures known in the art (see, for example, Sambrook et al., Molecular Cloning, A Laboratory Manual, 2^{nd} edition, Cold Spring Harbor Laboratory Press, Cold Spring Harbor, N.Y., 1989, which is incorporated herein by reference). The transducer of MAS-signalling is then expressed by the cultured cells, and selected agents are screened for the desired effect on the cell. Means for amplifying nucleic acid sequences which may be employed to amplify sequences encoding the signalling protein or portions thereof are described in U.S. Pat. Nos. 4,683,195 and 4,683,202, incorporated herein by reference.

In yet another aspect, the screening assays provided by the invention relate to transgenic mammals whose germ cells and somatic cells contain a nucleotide sequence encoding transducer of MAS-signalling or a selected portion of the transducer of MAS-signalling which, for example, contains regulatory domains. In yet a further aspect, the screening assays provided by the invention relate to transgenic mammals where the nucleotide sequence encoding a transducer of MAS-signalling is molecularly targeted to produce knock out animals with the phenotypical loss of the specific MAS signalling function. Preferentially, the molecular knock out is tissue specific to gonadal tissue (ovary or testes) and is timely controlled in the development, thus inducible. There are several means by which a sequence encoding, for example, the human transducer of MAS-signalling may be introduced into a non-human mammalian embryo or, alternatively, knocked out, some of which are described in, for example, U.S. Pat. No. 4,736,866, Jaenisch, Science 240: 1468-1474 (1988) and Westphal et al., Annu. Rev. Cell Biol. 5: 181-196 (1989), which are incorporated herein by reference. The animal's cells then express the signalling protein and thus may be used as a convenient model for testing or screening selected agonists or antagonists.

In another aspect the invention concerns diagnostic methods and compositions. By means of having the transducer of MAS-signalling molecule and antibodies thereto, a variety of diagnostic assays are provided. For example, with antibodies, including monoclonal antibodies, to the transducer of MAS-signalling, the presence and/or concentration of signalling protein in selected cells or tissues in an individual or culture of interest may be determined. These assays can be used in the diagnosis and/or treatment of diseases such as, for example, male infertility, female infertility, or by means of contraception in both gender.

Numerous types of immunoassays are available and are known to those skilled in the art, for example, competitive assays, sandwich assays, and the like, as generally described in, for example U.S. Pat. Nos. 4,642,285; 4,376,110; 4,016,043; 3,879,262; 3,852,157; 3,850,752; 3,839,153; 3,791,932; and Harlow and Lane, Antibodies, A Laboratory Manual, Cold Spring Harbor Publications, N.Y. (1988), each incorporated by reference herein. In one assay format, the transducer of MAS-signalling is identified and/or quantified by using labelled antibodies, preferably monoclonal antibodies which are reacted with brain tissues, for example, ovarian or testicular tissue, oocyte preparations, or semen samples, and determining the specific binding thereto, the assay typically being performed under conditions conducive to immune complex formation. Unlabeled primary antibody can be used in combination with labels that are reactive with primary antibody to detect the signalling protein. For example, the primary antibody may be detected indirectly by a labelled secondary antibody made to specifically detect the primary antibody. Alternatively, the anti-transducer of MAS-signalling-antibody can be directly labelled. A wide variety of labels may be employed, such as radionuclides, particles (for example, gold, ferritin, magnetic particles, red blood cells), flourophores, chemiluminescers, enzymes, enzyme substrates, enzyme cofactors, enzyme inhibitors, and ligands (particularly haptens).

The RNA encoding the transducer of MAS-signalling may be directly detected in cells with a labelled synthetic oligonucleotide probe targeting the transducer of MAS-signalling RNA in a hybridisation procedure. Also, the polymerase chain reaction (Saiki et al., Science 239 (1988), 487, and U.S. Pat. No. 4,683,195, each reference is hereby incorporated by reference) may be used to amplify DNA sequences, which are subsequently detected by their characteristic size on agarose gels, Southern blot of these gels using the transducer of MAS-signalling DNA or a oligonucleotide probe, or a dot blot using similar probes. The probes may comprise from about 14 nucleotides to about 25 or more nucleotides, preferably, 40 to 60 nucleotides, and in some instances a substantial portion or even the entire cDNA of the transducer of MAS-signalling may be used. The probes are labelled with detectable signal, such as an enzyme, biotin, a radionuclide, fluorophore, chemiluminescer, and paramagnetic particle. High stringency in connection with hybridisation is obtained using the proper temperature and salt concentration.

Kits can also be supplied for use with the signalling protein of the subject invention in the detection of the presence of the signalling protein or antibodies thereto, as might be desired in the case of autoimmune disease. Thus, antibodies to the transducer of MAS-signalling, preferably monospecific antibodies such as monoclonal antibodies, or compositions of the signalling protein may be provided, usually in lyophilised form in a container, either segregated or in conjunction with additional reagents, such as anti-antibodies, labels, gene probes, polymerase chain reaction primers and polymerase, and the like.

Even more specifically, the present invention relates to an isolated and/or purified polynucleotide molecule which hybridises at high stringency to an oligonucleotide or polynucleotide of 25 or more contiguous nucleotides of SEQ ID NO: 1 or SEQ ID NO: 3 and which polynucleotide codes for a) a transducer of MAS-signalling; or b) a regulatory domain of a transducer of MAS-signalling. This polynucleotide may be a RNA antisense sequence or a cDNA sequence. This polynucleotide may encode a polypeptide displaying Transducer of MAS-signalling activity. This polynucleotide may encode a transducer of MAS-signalling having the amino acid sequence of SEQ ID NO: 2 or SEQ ID NO: 4. The polynucleotide may have the nucleotides of SEQ ID NO: 1 or SEQ ID NO: 3.

Furthermore, this invention relates to a probe of at least 12 nucleotides, said probe being capable of hybridising with nucleic acids which encode a Transducer of MAS-signalling. This probe may comprise an oligonucleotide or polynucleotide of 25 or more contiguous nucleotides of SEQ ID NO: 1 or SEQ ID NO: 3 capable of specifically hybridising with a gene which encodes a transducer of MAS-signalling, or allelic and species variants thereof. This probe may comprise from about 40 to about 60 nucleotides in length. This probe may be labelled to provide a detectable signal. This probe may comprise the nucleotides of SEQ ID NO: 1 or SEQ ID NO: 3.

Furthermore, the present invention relates to a DNA construct comprising a DNA sequence which hybridises at high stringency to an oligonucleotide or polynucleotide of 25 or more contiguous nucleotides of SEQ ID NO: 1 or SEQ ID NO: 3 and which encodes a) a transducer of MAS-signalling; or b) a regulatory domain of a transducer of MAS-signalling. This DNA construct may have a DNA sequence encoding a transducer of MAS-signalling having the amino acid sequence of SEQ ID NO: 2 or SEQ ID NO: 4.

Furthermore, the present invention relates to a cultured cell line, yeast or bacteria transformed or transfected with a DNA construct which comprises a DNA sequence which hybridises at high stringency to an oligonucleotide or polynucleotide of 25 or more contiguous nucleotides of SEQ ID NO: 1 or SEQ ID NO: 3 and which encodes a) a transducer of MAS-signalling; or b) a regulatory domain of a transducer of MAS-signalling. This cell line, yeast or bacteria may not express endogenous transducers of MAS-signalling.

The transducer of MAS-signalling, a peptide fragment thereof or a salt thereof according to the present invention may be isolated and/or purified. The isolated and/or purified protein (transducer of MAS-signalling) may comprise the amino acid sequence of SEQ ID NO: 2 or SEQ ID NO: 4.

Furthermore, the present invention relates to an isolated antibody which specifically binds to a transducer of MAS-signalling. In this isolated antibody said antibody may be a monoclonal antibody. This isolated antibody may block the MAS stimulated signal transduction.

Furthermore, the present invention relates to a hybridoma which produces a monoclonal antibody as mentioned herein.

Furthermore, the present invention relates to a method for detecting the presence of a compound or a salt thereof which has affinity for a transducer of MAS-signalling, comprising the steps of a) contacting the compound with the transducer of MAS-signalling, a peptide fragment thereof or a salt thereof; and b) measuring the affinity of said compound for the transducer of MAS-signalling. This method for detecting the presence of transducer of MAS-signalling antagonists, may comprise the steps of a) exposing a compound in the presence of a transducer of MAS-signalling agonist to a transducer of MAS-signalling coupled to a response pathway under conditions and for a time sufficient to allow binding of the compound to the transducer of MAS-signalling and an associated response through the pathway; and b) detecting a reduction in the stimulation of the response pathway resulting from the binding of the compound to the transducer of MAS-signalling, relative to the stimulation of the response pathway by the transducer of MAS-signalling agonist alone and there from determining the presence of a transducer of MAS-signalling antagonist. Furthermore, a method for detecting the presence of transducer of MAS-signalling agonists, may comprise the steps of a) exposing a compound in the presence of a transducer of MAS-signalling antagonist to a transducer of MAS-signalling coupled to a response pathway under conditions and for a time sufficient to allow binding of the compound to the transducer of MAS-signalling and an associated response through the pathway; and b) detecting an increase of the stimulation of the response pathway resulting from the binding of the compound to the transducer of MAS-signalling, relative to the stimulation of the response pathway by the transducer of MAS-signalling antagonist alone and there from determining the presence of a transducer of MAS-signalling agonist.

Furthermore, the present invention relates to a compound or a salt thereof which has affinity for the transducer of MAS-signalling and which compound or salt is detected by a method described herein.

Furthermore, the present invention relates to a method for producing a transducer of MAS-signalling having the amino acid sequence of SEQ ID NO: 2 or SEQ ID NO: 4, which may comprise a) growing cells, yeast or bacteria transformed or transfected with a DNA construct which comprises a DNA sequence of SEQ ID NO: 1 or SEQ ID NO: 3 coding for the expression of the transducer of MAS-signalling, and b) isolating the transducer of MAS-signalling from the cells. In this method, the transducer of MAS-signalling may be isolated by immunoaffinity purification.

Furthermore, the present invention relates to a kit for screening a compound or a salt thereof which has affinity for a transducer of MAS-signalling, which contains the transducer of MAS-signalling, the peptide fragment thereof or a salt thereof.

The transducer of MAS-signalling according to the present invention may be a soluble and purified protein which is present in a buffer suitable for detecting ligands, for example by a binding assay. The transducer of MAS-signalling being a soluble and purified protein which is present in a buffer suitable for detecting ligands, for example by a binding assay, may be different from the amino acid sequence in SEQ ID NO: 6 and 8.

Furthermore, the present invention relates to a DNA construct which comprises a DNA sequence encoding a transducer of MAS-signalling as described herein or a DNA sequence coding for a functional analog thereof. The DNA construct comprises a DNA sequence encoding a transducer of MAS-signalling as defined herein or a DNA sequence coding for a functional analog thereof responding to FF-MAS may be different from the nucleotides of SEQ ID NO: 5 and 7. The DNA construct of the present invention may comprise the DNA sequence shown in SEQ ID NO: 1 or a fragment thereof, or a DNA sequence coding for a functional analogue thereof. This DNA construct comprising the DNA sequence shown in SEQ ID NO: 1 or a fragment thereof, or a DNA sequence coding for a functional analogue thereof, may be different from the nucleotides of SEQ ID NO: 5 and 7. The DNA construct according to the present invention may comprise the partial DNA sequence shown in SEQ ID NO: 5, or a DNA sequence coding for a functional analogue thereof. This DNA construct comprising the partial DNA sequence shown in SEQ ID NO: 5, or a DNA sequence coding for a functional analogue thereof may be different from the nucleotides of SEQ ID NO: 5 and 7.

Furthermore, the present invention relates to a recombinant expression vector which carries an inserted DNA construct according to any one of the preceding claims to a DNA construct.

Furthermore, the present invention relates to a cell containing a recombinant expression vector as defined herein. This cell may contain a DNA construct as defined herein integrated in its genome. This cell may be a eukaryotic cell, in particular an insect or a mammalian cell.

Furthermore, the present invention relates to a method of screening for ligands to the transducer of MAS-signalling, i.e., agonists or antagonists of FF-MAS activity, the method comprising incubating a transducer of MAS-signalling as defined herein with a substance suspected to be an agonist or antagonist of FF-MAS activity, and subsequently with FF-MAS, or an analogue thereof, and detecting any function of FF-MAS, or the analogue to the transducer of MAS-signalling. Alternatively, the method of screening for ligands to the Transducer of MAS-signalling, i.e., agonists or antagonists of FF-MAS activity, may comprise incubating FF-MAS, or an analogue thereof with a substance suspected to be an agonist or antagonist of activity of FF-MAS, and subsequently with a transducer of MAS-signalling as described herein, and detecting any function of FF-MAS, or the analogue to the transducer of MAS-signalling.

Furthermore, the present invention relates to the use of a transducer of MAS-signalling as defined herein for screening for agonists or antagonists of activity of FF-MAS.

Furthermore, the present invention relates to the use of DNA constructs as defined herein for isolation of tissue and/or organ specific variants of the transducer of MAS-signalling.

Furthermore, the present invention relates to the use of a transducer of MAS-signalling isolated as described herein.

For the sake of completeness, it is hereby noticed that this invention does not relate to anything known. In some countries, this applies analogously to that mentioned specifically in our international application No.: PCT/DK 01/00550 (WO 02/16433) published after the priority date of this application. WO 02/16433 is hereby incorporated by reference. SEQ ID NO: 1-14 herein are identical to SEQ ID NO: 1-14, respectively, in WO 02/16433.

The following examples are offered by way of illustration, not by limitation.

### EXAMPLE 1

Microinjection of Phosphorothionate Oligonucleotides into Mouse Oocytes

Two antisense oligonucleotides (20 nucleotides) were utilized for microinjection: 5′-TCCACGATGGACGCCATCTT-3′ and 5′-GCCAGCAGGAGAGCCATTCG-3′, complementary to the kozak sequence of the mRNA encoded by the cDNA sequence herein designated SAM1a and SAM1b, respectively, both of which are defined in SEQ ID NO: 1 and SEQ ID NO: 3, respectively, shown below. In control experiments, the corresponding sense oligonucleotides were microinjected: 5′-AAGATGGCGTCCATCGTGGA-3′ and 5′-CGAATGGCTCTCCTGCTGGC-3′ for mRNA SAM1a and SAM1b, respectively. SAM1a antisense was co-injected with SAM1b antisense from a stock solution containing 1.25 μg/μl of each nucleotide in 10% human serum albumin (hereinafter designated HSA) plus 5 mM Tris (pH value: 7.5). SAM1a sense was co-injected with SAM1b sense from a stock solution containing 1.25 μg/μl of each nucleotide in 10% HSA plus 5 mM Tris (pH value: 7.5). Approximately 12 pg of each oligonucleotide (10 pl) were injected into the cytoplasma of individual germinal vesicle (GV)-stage oocytes loaded in a droplet of alpha-MEM supplemented with 0.8% HSA and 3 mM hypoxantine under mineral oil in a 35 mm petri dish on the stage of an inverted microscope. The oocytes were obtained from the ovaries of 21-24 days old mice following 48 hours priming with follicle stimulating hormone (hereinafter designated FSH) as described by Grøndahl et al. 1998 in Biol. Reprod. 58 (1998), 1297 et seq. Oocytes were sucked on to a holding pipette (120 μM outer diameter and 20 μm inner diameter) and an injection pipette (Eppendorph, Hamburg, Germany) was fitted to a pressure microinjector (Eppendorph, Hamburg, Germany). The pipette holder was attached to a piezoelectric positioning system (Burleigh, N.Y., USA) mounted on a motorized micromanipulator (Luigs and Neumann, Ratingen, Germany). The injection pipette was pushed against the zona pelludica, and then a piezoelectric pulse was given, moving the injection pipette 20 μm forward. During this movement the pipette penetrated the zona pelludica and oolema and then a brief pressure pulse was applied to release a volume of approximately 10 μl into the oocytes cytoplasm. Injected oocytes were placed in a CO₂ incubator at 37° C. for 20 hours before resumption of meiosis was triggered by addition of 10 μM FF-MAS to the hypoxanthine containing medium. The effect of FF-MAS was evaluated after 24 hours of further incubation as the number of oocytes in germinal vesicle breakdown (hereinafter designated GVBD). The rationale for the 20 hours cultivation period following injection of antisense oligonucleotides is to allow for degradation of mRNA coding for SAM1a and SAM1b protein. Consequently, when the level of MAS receptor protein or Transducer of MAS-signalling is reduced in the oocytes, the MAS response is blunted (from 100% to 50%, vide the table below).

TABLE 1

| | GVBD/GVBD + GV |
|---|---|
| Oligonucleotide | 10 μM FF-MAS (24 hours) |
| SAM1a + SAM1b | 13/25 |
| Antisense | |
| SAM1a + SAM1b | 10/10 |
| Sense | |
| Non-injected oocytes | 26/29 |

As shown in Table 1, GVBD was inhibited by 50% in antisense injected oocytes compared to control (i.e., sense injected and non-injected oocytes). This result indicates a selective inhibition of the mRNAs coding for SAM1a and SAM1b by the antisense probe. Furthermore, these results indicate that SAM1a and SAM1b proteins are crucial involved in the MAS signalling, since a functional knock out of de novo protein synthesis of these molecules partly disrupt the MAS signals in oocytes.

SAM1a and SAM1b are two closely related proteins originating from the same gene which possesses complementary functions regarding MAS signalling in oocytes.

### EXAMPLE 2

Cloning of SAM1

A cDNA library was prepared from mRNA isolated from 10,000 oocytes, from 24 days old mice. The cDNA library was constructed in the pSPORT plasmid vector (Life Technologies). Clones were picked at random and partially sequenced, and the sequences were assembled using phred/phrap programs. Out of several thousand clones that were sequenced, an assembly of two exhibited 21% amino acids identity to a human Oxysterol Binding Protein. The longest clone MOCY2864 was completely sequenced and no identical or orthologous genes were found in the databases. This new gene was named SAM1. Amplification of the 5′ end of SAM1 cDNA was performed by PCR on the oocyte library using a primer specific for pSPORT, #176959 (SEQ ID NO: 9) and a primer specific for SAM1 #198241 (SEQ ID NO: 10).

This revealed cDNAs with two different 5′ ends, which were designated SAM1a and SAM1b. Full-length PCR amplification was done on the mouse oocyte library using primer #199772 (SEQ ID NO: 11) and #198239 (SEQ ID NO: 12) for SAM1a and #201790 (SEQ ID NO: 13) and #198239 (SEQ ID NO: 12) for SAM1b. Recognition sites for NheI and NotI, respectively, were incorporated in the primers. SAM1a and SAMb cDNAs were digested with NheI and NotI restriction enzymes and cloned into pcDNA3,1+ (Invitrogen).

DNA constructs directing the expression of SAM1a and SAM1b proteins fused to a C-terminal histidine stretch, which could be used for in purification because of its affinity to nickel-columns were made as follows. SAM1a and SAM1b cDNAs were PCR amplified using the primers #199772 (SEQ ID NO: 11) and #211465 (SEQ ID NO: 14) and primers #201790 (SEQ ID NO: 13) and #211465 (SEQ ID NO: 14), respectively. Recognition sites for NheI and XmaI, respectively, were incorporated in the primers. The PCR-products were then cloned into pBlueBac4,5V5HIS (Invitrogen) using the restriction enzymes NheI and XmaI. This intermediate construct was then digested by SmaI and BstBI, filled-in using the Klenow fragment of DNA polymerase1 and then religated. These construct were designated “SAM1a in pBlueBac4,5V5HIS” and “SAM1b in pBlueBac4,5V5HIS”.

SAM1 Expression in Sf9 cells

SAM1a-HIS and SAM1b-HIS proteins were expressed using recombinant Baculo virus in Sf9 cells according to the “Bac-N-Blue™ Transfection Kit” manual (Invitrogen).

To infect Sf9 insect cells 0,5 μg Bac-N-Blue™ DNA and the recombinant transfer plasmid “SAM1 in pBlueBac4,5V5HIS” (4 μg) were incubated with 1 ml Grace's Insect Media and and 20 μl Insectin-Plus Liposomes for 15 minutes, then the mixture was added to 2×10⁶ Sf9 cells in a 60 mm dish. The cells were left for 96 hours rocking at 27° C. Vira were isolated and plaque assay was performed. Putative recombinant plaques were picked and P-1 viral stocks were made. PCR analysis of recombinant viral clones was done and from positive clones high-titer viral stocks were then prepared. For large-scale SAM1a and SAM1b expression 500 ml Sf9 cells (2,0×10⁶ cells/ml) was infected with 25 ml virus (1,8×108 plaque forming units/ml) or 60 ml virus (6×10⁷ pfu/ml) for SAM1a and SAM1b, respectively. After 70 hours of incubation the cells were pelleted by centrifugation and the protein was purified.

### Example 3

Purification of SAM1a and SAM1b

Purification of HIS-SAM1a and HIS-SAM1b was performed according to the manual of QIAGEN: The QIAexpressionist forth edition.

Briefly, cell cultures of SF9 insect cells (containing the construct for 6xHis-SAM1a or 6xHis-SAM1b in a baculovirus expression vector) were centrifuged, pellets were lysed by addition of lysis buffer (50 mM NaH₂PO₄, 300 mM NaCl, 10 mM imidazole, pH 8) and lysozyme, sonication on ice 6×10 seconds, where after the lysates were cleared by centrifugation. Cleared lysates were incubated under mild agitation with 50% slurry of Ni-NTA agarose (QIAGEN), for binding of 6xHis-SAM1a, washed twice (50 mM NaH₂PO₄, 300 mM NaCl, 20 mM imidazole, pH 8) and eluted with high concentration of imidazole (50 mM NaH₂PO₄, 300 mM NaCl, 250 mM imidazole, pH 8). Purified proteins were analysed on Coomassie (Brilliant Blue, Sigma) stained SDS-polyacrylamide gels (NuPAGE 4-12% Bis-Tris gel, Invitrogen) for evaluation of yield and purity.

What is claimed is:

1. An isolated nucleic acid molecule which comprises a nucleotide sequence that A) hybridises at high stringency to a probe having a sequence of 25 or more contiguous nucleotides of SEQ ID NO: 1 or SEQ ID NO: 3 and B) encodes a) a transducer of meiosis activating sterol (MAS)-signalling activity; or b) a regulatory domain of a transducer of MAS-signalling.

2. The nucleic acid molecule of claim 1, wherein said nucleotide sequence of said molecule is an RNA antisense sequence.

3. The nucleic acid molecule of claim 1, wherein said nucleotide sequence of said molecule is a cDNA sequence.

4. The nucleic acid molecule of claim 1, wherein said nucleotide sequence of said molecule encodes a polypeptide that is a transducer of MAS-signalling activity.

5. The nucleic acid molecule of claim 1, wherein said nucleotide sequence of said molecule encodes a transducer of MAS-signalling having the amino acid sequence of SEQ ID NO: 2 or SEQ ID NO: 4.

6. The nucleic acid molecule of claim 1, wherein said nucleic acid molecule has a nucleotide sequence according to SEQ ID NO: 1 or SEQ ID NO: 3.

7. An isolated nucleic acid molecule comprising at least 12 contiguous nucleotides, where said molecule is capable of hybridising with a nucleic acid molecule that encodes a transducer of MAS-signalling.

8. The nucleic acid molecule of claim 7, wherein said molecule comprises 25 or more contiguous nucleotides of SEQ ID NO: 1 or SEQ ID NO: 3 that are capable of specifically hybridising with a gene which encodes a transducer of MAS-signalling.

9. The nucleic acid molecule of claim 7, wherein said molecule comprises from about 40 to about 60 nucleotides.

10. The nucleic acid molecule of claim 7, wherein said molecule is labelled to provide a detectable signal.

11. The nucleic acid molecule of claim 7, wherein said molecule comprises SEQ ID NO: 1 or SEQ ID NO: 3.

12. An expression vector comprising a nucleotide sequence which hybridises at high stringency to aprobe having a sequence of 25 or more contiguous nucleotides of SEQ ID NO: 1 or SEQ ID NO: 3 and which encodes a) a transducer of MAS-signalling; or b) a regulatory domain of a transducer of MAS-signalling.

13. The vector of claim 12, wherein the nucleotide sequence encodes a transducer of MAS-signalling having an amino acid sequence according to SEQ ID NO: 2 or SEQ ID NO: 4.

14. A cell line, yeast or bacteria which contains an expression vector that comprises a nucleotide sequence which hybridises at high stringency to a probe having a sequence of 25 or more contiguous nucleotides of SEQ ID NO: 1 or SEQ ID NO: 3 and which encodes a) a transducer of MAS-signalling; or b) a regulatory domain of a transducer of MAS-signalling.

15. The cell line, yeast or bacteria of claim 14, wherein said cell line, yeast or bacteria does not express endogenous transducers of MAS-signalling.

16. An isolated transducer of MAS-signalling, a peptide fragment thereof or a salt thereof.

17. An isolated antibody which specifically binds to a transducer of MAS-signalling.

18. The isolated antibody of claim 17 wherein said antibody is a monoclonal antibody.

19. The isolated antibody of claim 17, wherein said antibody blocks the signal transduction pathway of FF-MAS.

20. A hybridoma which produces a monoclonal antibody according to claim 17.

21. A method for detecting the presence of a compound or a salt thereof which has affinity for a transducer of MAS-signalling, said method comprising a) contacting the compound with the transducer of MAS-signalling, a peptide fragment thereof or a salt thereof; and b) measuring the affinity of said compound for the transducer of MAS-signalling.

22. A method for detecting the presence of an antagonist of a transducer of MAS-signalling, said method comprising a) exposing a compound in the presence of an agonist of a transducer of MAS-signalling to a transducer of MAS-signalling coupled to a response pathway under conditions sufficient and for a time to allow binding of the compound to the transducer of MAS-signalling and an associated response through the pathway; and b) detecting a reduction in the stimulation of the response pathway resulting from the binding of the compound to the transducer of MAS-signalling relative to the stimulation of the response pathway by the transducer of MAS-signalling agonist alone, and therefrom determining the presence of an antagonist of a transducer of MAS-signalling.

23. A method for detecting the presence of an agonist of a transducer of MAS-signalling, said method comprising a) exposing a compound in the presence of a MAS antagonist to a transducer of MAS-signalling coupled to a response pathway under conditions sufficient and for a time to allow binding of the compound to the transducer of MAS-signalling and an associated response through the pathway; and b) detecting an increase of the stimulation of the response pathway resulting from the binding of the compound to the transducer of MAS-signalling relative to the stimulation of the response pathway by the transducer of MAS-signalling antagonist alone, and therefrom determining the presence of an agonist of a transducer of MAS-signalling.

24. A compound or a salt thereof which has affinity for the transducer of MAS-signalling and which compound or salt is detected by the method according to claim 21.

25. A method for producing a transducer of MAS-signalling having the amino acid sequence of SEQ ID NO: 2 or SEQ ID NO: 4, said method comprising a) growing cells, yeast or bacteria containing an expression vector which comprises a nucleotide sequence according to SEQ ID NO: 1 or SEQ ID NO: 3 under conditions sufficient to express said transducer of MAS-signalling, and b) isolating the transducer of MAS-signalling expressed from said cells.

26. The method of claim 25, wherein the expressed transducer of MAS-signalling is isolated by immunoaffinity purification.

27. A kit for screening for a compound or a salt thereof which has affinity for a transducer of MAS-signalling, said kit comprising a transducer of MAS-signalling, a peptide fragment thereof or a salt thereof.

28. A transducer of MAS-signalling which is a soluble and purified protein and which is present in a buffer suitable for detecting ligands.

29. The transducer of MAS-signalling according to claim 28, wherein said transducer has an amino acid sequence that it is different from the amino acid sequence in SEQ ID NO: 6 or SEQ ID NO:8.

30. An expression vector which comprises a nucleotide sequence encoding a transducer of MAS-signalling or a functional analog thereof.

31. The expression vector according to claim 30, wherein said expression vector comprises a nucleotide sequence that it is different from the nucleotide sequence of SEQ ID NO: 5 or SEQ ID NO: 7.

32. An expression vector comprising a nucleotide sequence according to SEQ ID NO: 1 or a fragment or functional analogue thereof.

33. The expression vector of claim 32, wherein said vector comprises a nucleotide sequence that it is different from the nucleotide sequence of SEQ ID NO: 5 or SEQ ID NO: 7.

34. An expression vector which comprises a nucleotide sequence according to SEQ ID NO: 5 or a functional analogue thereof.

35. The expression vector of claim 34, wherein said vector comprises a nucleotide sequence that it is different from the nucleotide sequence of SEQ ID NO: 5 or SEQ ID NO: 7.

36. A recombinant expression vector which carries an inserted DNA construct according to any one of the preceding claims to a DNA construct.

37. A cell containing an expression vector according to claim 12.

38. The cell of claim 37, wherein said expression vector is integrated in its genome.

39. The cell of claim 37, wherein said cell is a eukaryotic cell.

40. A method for detecting the presence of an agonist or antagonist of a transducer of MAS-signalling, said method comprising a) incubating a transducer of MAS-signalling with a substance suspected to be an agonist or antagonist of transducer of MAS-signalling, and subsequently with FF-MAS or an analogue thereof, and b) detecting any signalling event of FF-MAS or the analogue to the transducer of MAS-signalling and therefrom detecting the presence of said agonist or antagonist.

41. A method for detecting the presence of an agonist or antagonist of a transducer of MAS-signalling, said method comprising a) incubating FF-MAS or an analogue thereof with a substance suspected to be an agonist or antagonist of activity of the transducer of MAS-signalling, and subsequently with a transducer of MAS-signalling, and b) detecting any signalling event of FF-MAS or the analogue thereof to the transducer of MAS-signalling and therefrom detecting the presence of said agonist or antagonist.

42. Use of a transducer of MAS-signalling according to any one of the preceding claims to a transducer of MAS-signalling for screening for agonists or antagonist of activity of FF-MAS.

43. Use of DNA constructs according to any one of the preceding claims to a DNA construct for isolation of tissue and/or organ specific variants of the transducer of MAS-signalling according to any one of the preceding claim to a transducer of MAS-signalling.

44. Use of a transducer of MAS-signalling isolated according to the preceding claim for the screening of MAS agonists or antagonist.

45. Any novel feature or combination of features described herein.

46. A compound or a salt thereof which has affinity for the transducer of MAS-signalling and which compound or salt is detected by the method according to claim 22.

47. A compound or a salt thereof which has affinity for the transducer of MAS-signalling and which compound or salt is detected by the method according to claim 23.

48. The cell of claim 39, wherein said eukaryotic cell is an insect cell or a mammalian cell


SEQUENCE LISTING (18 sequences)

SEQ ID NO: 1 - DNA, 2814 bp, mouse
  CDS (26)..(2194)
tttgttgtcattggcggctcccaagatggcgtccatcgtggaagggccgctgagcaaatggactaacgtgatgaagggatggcagtatcgttggttcgtgctggactacaatgcagggctgctctcctactacacgtccaaggacaaaatgatgagaggctctcgaagaggatgcgttagactcagaggagctgtgattggtatagacgacgaggacgacagcaccttcacaatcactgtcgatcagaaaaccttccacttccaggctcgagatgcagacgagcgagagaagtggatccatgccttagaagaaactattcttcgccatactcttcagcttcaaggtttggattcaggattcatccccagtgtccaagactttgataagaaacttaccgaggctgacgcgtacctgcagatcttgatagaacaattaaagctttttgatgacaagcttcaaaattgtaaagatgatgaacagagaaagaaagttgaaactctcaaagacacaacaaatagcatggtagaatcaattaaacactgcattgtgttgctacagattgctaaaagtactattaatcctgtagatgcaatataccagcctagtcccttggaacctgtgatcagcacaatgccttcccagactgccttacctccagaacccgctcagttgtgtaagtcagagcagcgtccatcttccttacctgttggacctgtgttagctaccttgggacatcatcagactccaacaccaaatagtacaggcagtgggaactcaccacctagcagcagtctgactcctcccagccatgtcaacttgtctccaaatacagtcccagagttctcttactctagcagtgaagatgagttctatgatgctgatgaattccatcaaagtggctcgtccccaaagcgcttaatagattcttctggatctgcctcagtcttgacacacagcagctccggaaatagcttaaaacgcccagataccacagagtctctgaattcctccatgtccaatggcacaagcgatgctgatctttttgactcacatgacgacagagatgatgatggggaggctgggtcagtggaggagcacaagagcgttatcatgcacctcttatcacaagtcaggctggggatggacctcacaaaggtagttcttccaacgtttattctcgagagaagatctctgttagaaatgtatgcagactttttcgcacatccagacctgttcgtgagcattagtgatcagaaggatcccagggatcgaatggttcaggttgtgaaatggtacctctcggccttccatgcaggaaggagaggatcggtggccaaaaagccgtacaatcctattttgggtgagatctttcagtgtcactggacgttgccgaatgatactgaagagaacgcagagctcgtttcagaagggccggttccctgggtttctaagaacagtgtaacatttgtggctgagcaagtttcccaccatccgcccatttcagccttttatgctgagtgttttaacaagaagatacaattcaatgctcatatctggactaaatcaaaattccttgggatgtcaattggggtacacaacataggtcagggctgtgtctcgtgtctggagtacgatgagcactacatcctcacgttccccaatggctatggaaggtctatcctgacagtgccctgggtggaattgggaggagaatgcaatatcaactgctccaaaacgggttacagcgcaaacatcgtcttccacactaagcctttctatgggggcaagaagcacagaattactgcagagattttttctccgaatgacaagaaatccttctgctcaattgaaggggaatggaatggtatcatgtatgcaaaatacgcaacaggggaaaacactgtctttgtagacaccaagaaattgcctataatcaagaaaaaggtgaggaagttggaagatcagaatgagtatgagtcccgcagcctttggaaggatgtcactttcaatttaaaaatcagagacattgatgcagcaacggaagcaaagcacagacttgaagaaagacaaagagcagaagcccgagaaaggaaggagaaggaaattcagtgggagacgaggctctttcacgaggatggcgaatgctgggtttacgatgagcctttactgaagcgtcttggtgctgtgaagcattaggccgacaaccgagtccacacctggtgaccagggcagtaggcgtaattaagcaacaatcgatcttccttcaggagagcttgtcacttccttcttaacgcagtggttcctatctcagggatactggacttgacgacacagatgaacaattaaagtggaaaccgcttcccttttctcctctgtggcagttacaattttgacttcaggcctgagaaaaacttcaggtttcgaaacatgacatctcttccttttccagatcccatgctttgaaaaatatttatagacagttccaggtctcagcttcctgtcctctagttctgctgttcgggcataaaatctttatctccagttcatataatcttgagttttagatakacacacatgcgtaacagctgacagtttttcacaagtacacccacctgtaaatactgtakcctcagtttagaaaattagtgcatgtatgaaaatcaagtgttaggaaatttcatggtttcacctataacctttattttagaattgaactatgattagattgtatctaaacctgaagtataattatatgcagtgcttcttaaggcttcataaaataattttccaacctttttaaaaaaaaaaaaaaa

SEQ ID NO: 2 - protein, 723 aa, mouse
MASIVEGPLSKWTNVMKGWQYRWFVLDYNAGLLSYYTSKDKMMRGSRRGCVRLRGAVIGIDDEDDSTFTITVDQKTFHFQARDADEREKWIHALEETILRHTLQLQGLDSGFIPSVQDFDKKLTEADAYLQILIEQLKLFDDKLQNCKDDEQRKKVETLKDTTNSMVESIKHCIVLLQIAKSTINPVDAIYQPSPLEPVISTMPSQTALPPEPAQLCKSEQRPSSLPVGPVLATLGHHQTPTPNSTGSGNSPPSSSLTPPSHVNLSPNTVPEFSYSSSEDEFYDADEFHQSGSSPKRLIDSSGSASVLTHSSSGNSLKRPDTTESLNSSMSNGTSDADLFDSHDDRDDDGEAGSVEEHKSVIMHLLSQVRLGMDLTKVVLPTFILERRSLLEMYADFFAHPDLFVSISDQKDPRDRMVQVVKWYLSAFHAGRRGSVAKKPYNPILGEIFQCHWTLPNDTEENAELVSEGPVPWVSKNSVTFVAEQVSHHPPISAFYAECFNKKIQFNAHIWTKSKFLGMSIGVHNIGQGCVSCLEYDEHYILTFPNGYGRSILTVPWVELGGECNINCSKTGYSANIVFHTKPFYGGKKHRITAEIFSPNDKKSFCSIEGEWNGIMYAKYATGENTVFVDTKKLPIIKKKVRKLEDQNEYESRSLWKDVTFNLKIRDIDAATEAKHRLEERQRAEARERKEKEIQWETRLFHEDGECWVYDEPLLKRLGAVKH

SEQ ID NO: 3 - DNA, 2534 bp, mouse
  CDS (37)..(1914)
gggcccctcccctgagacggcggccggccgccgcgaatggctctcctgctggccgcttgcggaggtttggattcaggattcatccccagtgtccaagactttgataagaaacttaccgaggctgacgcgtacctgcagatcttgatagaacaattaaagctttttgatgacaagcttcaaaattgtaaagatgatgaacagagaaagaaagttgaaactctcaaagacacaacaaatagcatggtagaatcaattaaacactgcattgtgttgctacagattgctaaaagtactattaatcctgtagatgcaatataccagcctagtcccttggaacctgtgatcagcacaatgccttcccagactgccttacctccagaacccgctcagttgtgtaagtcagagcagcgtccatcttccttacctgttggacctgtgttagctaccttgggacatcatcagactccaacaccaaatagtacaggcagtgggaactcaccacctagcagcagtctgactcctcccagccatgtcaacttgtctccaaatacagtcccagagttctcttactctagcagtgaagatgagttctatgatgctgatgaattccatcaaagtggctcgtccccaaagcgcttaatagattcttctggatctgcctcagtcttgacacacagcagctccggaaatagcttaaaacgcccagataccacagagtctctgaattcctccatgtccaatggcacaagcgatgctgatctttttgactcacatgacgacagagatgatgatggggaggctgggtcagtggaggagcacaagagcgttatcatgcacctcttatcacaagtcaggctggggatggacctcacaaaggtagttcttccaacgtttattctcgagagaagatctctgttagaaatgtatgcagactttttcgcacatccagacctgttcgtgagcattagtgatcagaaggatcccagggatcgaatggttcaggttgtgaaatggtacctctcggccttccatgcaggaaggagaggatcggtggccaaaaagccgtacaatcctattttgggtgagatctttcagtgtcactggacgttgccgaatgatactgaagagaacgcagagctcgtttcagaagggccggttccctgggtttctaagaacagtgtaacatttgtggctgagcaagtttcccaccatccgcccatttcagccttttatgctgagtgttttaacaagaagatacaattcaatgctcatatctggactaaatcaaaattccttgggatgtcaattggggtacacaacataggtcagggctgtgtctcgtgtctggagtacgatgagcactacatcctcacgttccccaatggctatggaaggtctatcctgacagtgccctgggtggaattgggaggagaatgcaatatcaactgctccaaaacgggttacagcgcaaacatcgtcttccacactaagcctttctatgggggcaagaagcacagaattactgcagagattttttctccgaatgacaagaaatccttctgctcaattgaaggggaatggaatggtatcatgtatgcaaaatacgcaacaggggaaaacactgtctttgtagacaccaagaaattgcctataatcaagaaaaaggtgaggaagttggaagatcagaatgagtatgagtcccgcagcctttggaaggatgtcactttcaatttaaaaatcagagacattgatgcagcaacggaagcaaagcacagacttgaagaaagacaaagagcagaagcccgagaaaggaaggagaaggaaattcagtgggagacgaggctctttcacgaggatggcgaatgctgggtttacgatgagcctttactgaagcgtcttggtgctgtgaagcattaggccgacaaccgagtccacacctggtgaccagggcagtaggcgtaattaagcaacaatcgatcttccttcaggagagcttgtcacttccttcttaacgcagtggttcctatctcagggatactggacttgacgacacagatgaacaattaaagtggaaaccgcttcccttttctcctctgtggcagttacaattttgacttcaggcctgagaaaaacttcaggtttcgaaacatgacatctcttccttttccagatcccatgctttgaaaaatatttatagacagttccaggtctcagcttcctgtcctctagttctgctgttcgggcataaaatctttatctccagttcatataatcttgagttttagatakacacacatgcgtaacagctgacagtttttcacaagtacacccacctgtaaatactgtakcctcagtttagaaaattagtgcatgtatgaaaatcaagtgttaggaaatttcatggtttcacctataacctttattttagaattgaactatgattagattgtatctaaacctgaagtataattatatgcagtgcttcttaaggcttcataaaataattttccaacctttttaaaaaaaaaaaaaaa

SEQ ID NO: 4 - protein, 626 aa, mouse
MALLLAACGGLDSGFIPSVQDFDKKLTEADAYLQILIEQLKLFDDKLQNCKDDEQRKKVETLKDTTNSMVESIKHCIVLLQIAKSTINPVDAIYQPSPLEPVISTMPSQTALPPEPAQLCKSEQRPSSLPVGPVLATLGHHQTPTPNSTGSGNSPPSSSLTPPSHVNLSPNTVPEFSYSSSEDEFYDADEFHQSGSSPKRLIDSSGSASVLTHSSSGNSLKRPDTTESLNSSMSNGTSDADLFDSHDDRDDDGEAGSVEEHKSVIMHLLSQVRLGMDLTKVVLPTFILERRSLLEMYADFFAHPDLFVSISDQKDPRDRMVQVVKWYLSAFHAGRRGSVAKKPYNPILGEIFQCHWTLPNDTEENAELVSEGPVPWVSKNSVTFVAEQVSHHPPISAFYAECFNKKIQFNAHIWTKSKFLGMSIGVHNIGQGCVSCLEYDEHYILTFPNGYGRSILTVPWVELGGECNINCSKTGYSANIVFHTKPFYGGKKHRITAEIFSPNDKKSFCSIEGEWNGIMYAKYATGENTVFVDTKKLPIIKKKVRKLEDQNEYESRSLWKDVTFNLKIRDIDAATEAKHRLEERQRAEARERKEKEIQWETRLFHEDGECWVYDEPLLKRLGAVKH

SEQ ID NO: 5 - DNA, 2840 bp, homosapien
  CDS (8)..(2179)
tcccaagatggcgtccatcatggaagggccgctgagcaaatggactaacgtgatgaagggctggcagtaccgttggttcgtgctggactacaatgcaggactgctctcctactacacgtccaaggacaaaatgatgagaggctctcgcagaggatgtgttagactcagaggagctgtgattggtatagacgatgaggacgacagcaccttcacaataactgttgatcagaaaaccttccatttccaggcccgtgatgctgatgagcgagagaagtggatccatgccttagaagaaacaattcttcgacatactctccagcttcaaggtttggattcaggatttgttcctagtgtccaagattttgataagaaacttacagaagctgatgcttacctacaaatcttgattgaacaattaaagctttttgatgacaagcttcaaaactgcaaagaagatgaacagagaaagaaaattgaaactctcaaagagacaacaaatagcatggtagaatcaattaaacactgcattgtgttgctgcagattgccaaaagtactattaatcccgtagatgcaatatatcaacctagtcctttggaacctgtgatcagcacaatgccttcccagactgtgttacctccagaacctgttcagttgtgtaagtcagagcagcgtccatcttccctaccagttggacctgtgttggctaccttgggacatcatcagactcctacaccaaatagtacaggcagtggccattcaccaccgagtagcagtctcacttctccaagccacgtgaacttgtctccaaatacagtcccagagttctcttactccagcagtgaagatgaattttatgatgctgatgaattccatcaaagtggctcatccccaaagcgcttaatagattcttctggatctgcctcagtcctgacacacagcagctcgggaaatagtctaaaacgcccagataccacagaatcacttaattcttccttgtccaatggaacaagtgatgctgacctgtttgattcacatgatgacagagatgatgatgcggaggcagggtctgtggaggagcacaagagcgttatcatgcatctcttgtcgcaggttagacttggaatggatcttactaaggtagttcttccaacgtttattcttgaaagaagatctcttttagaaatgtatgcagacttttttgcacatccggacctgtttgtgagcattagtgaccagaaggatcccaaggatcgaatggttcaggttgtgaaatggtacctctcagcctttcatgcgggaaggaaaggatcagttgccaaaaagccatacaatcccattttgggcgagatttttcagtgtcattggacattaccaaatgatactgaagagaacacagaactagtttcagaaggaccagttccctgggtttccaaaaacagtgtaacatttgtggctgagcaggtttcccatcatccacccatttcagccttttatgctgagtgttttaacaagaagatacaattcaatgctcatatctggaccaaatcaaaattccttgggatgtcaattggggtgcacaacatagggcagggctgtgtctcatgtctagactatgatgaacattacattctcacattccccaatggctatggaaggtctatcctcacagtgccctgggtggaattaggaggagaatgcaatattaattgttccaaaacaggctatagtgcaaatatcatcttccacactaaacccttctatgggggcaagaagcacagaattactgccgagattttttctccaaatgacaagaagtctttttgctcaattgaaggggaatggaatggtgtgatgtatgcaaaatatgcaacaggggaaaatacagtctttgtagataccaagaagttgcctataatcaagaagaaagtgaggaagttggaagatcagaacgagtatgaatcccgcagcctttggaaggatgtcactttcaacttaaaaatcagagacattgatgcagcaactgaagcaaagcacaggcttgaagaaagacaaagagcagaagcccgagaaaggaaggagaaggaaattcagtgggagacaaggttatttcatgaagatggagaatgctgggtttatgatgaaccattactgaaacgtcttggtgctgccaagcattaggttggaagatgcaaagtttatacctgatgatcagggcagtaggcataattcagcaacaaacaatcttcctttgggagaaacctgttcattccaatcttctaattacagtggttcctatctcagggatactggactttctgacgcagatgaacaattaaggggaaaagcttcccttttccctctgtggcagttacgattttgacttcagtcctgagaaaaacttcaggttttgaaaatcagatgatgtcttctccttttccaaacaccacacgttgaaagcatttataaatccaagtctgaaactctgcgctctagtactgctgttaagatacacaacttgtttcttagttcatataatctcgggatacacacacacacacatatatatatacacacacatacgtatacacacacatacatatataaatatacctgatgccagatttttttcataaatattctgcctactgtaaatatgggttcctctgagttgttttagaaaattagcgcaatgtattaaaatcaagtgttaggaaatttcatggtcttacctacaataacttttattttggaattgaactattattaaattgtatctaatcctggattacagtttaattaattattcttagtgcttaaggcttcataaagtaatttttcc

SEQ ID NO: 6 - protein, 723 aa, homosapien
MASIMEGPLSKWTNVMKGWQYRWFVLDYNAGLLSYYTSKDKMMRGSRRGCVRLRGAVIGIDDEDDSTFTITVDQKTFHFQARDADEREKWIHALEETILRHTLQLQGLDSGFVPSVQDFDKKLTEADAYLQILIEQLKLFDDKLQNCKEDEQRKKIETLKETTNSMVESIKHCIVLLQIAKSTINPVDAIYQPSPLEPVISTMPSQTVLPPEPVQLCKSEQRPSSLPVGPVLATLGHHQTPTPNSTGSGHSPPSSSLTSPSHVNLSPNTVPEFSYSSSEDEFYDADEFHQSGSSPKRLIDSSGSASVLTHSSSGNSLKRPDTTESLNSSLSNGTSDADLFDSHDDRDDDAEAGSVEEHKSVIMHLLSQVRLGMDLTKVVLPTFILERRSLLEMYADFFAHPDLFVSISDQKDPKDRMVQVVKWYLSAFHAGRKGSVAKKPYNPILGEIFQCHWTLPNDTEENTELVSEGPVPWVSKNSVTFVAEQVSHHPPISAFYAECFNKKIQFNAHIWTKSKFLGMSIGVHNIGQGCVSCLDYDEHYILTFPNGYGRSILTVPWVELGGECNINCSKTGYSANIIFHTKPFYGGKKHRITAEIFSPNDKKSFCSIEGEWNGVMYAKYATGENTVFVDTKKLPIIKKKVRKLEDQNEYESRSLWKDVTFNLKIRDIDAATEAKHRLEERQRAEARERKEKEIQWETRLFHEDGECWVYDEPLLKRLGAAKH

SEQ ID NO: 7 - DNA, 2457 bp, homosapien
  CDS (173)..(1849)
cggcccctacccctgagtccccggggtcccggccgccaggccggagcgcgaatgtcgtgctcaccctgcctccttcccgccgccccctgggctttttgatgacaagcttcaaaactgcaaagaagatgaacagagaaagaaaattgaaactctcaaagagacaacaaatagcatggtagaatcaattaaacactgcattgtgttgctgcagattgccaaaagtactattaatcccgtagatgcaatatatcaacctagtcctttggaacctgtgatcagcacaatgccttcccagactgtgttacctccagaacctgttcagttgtgtaagtcagagcagcgtccatcttccctaccagttggacctgtgttggctaccttgggacatcatcagactcctacaccaaatagtacaggcagtggccattcaccaccgagtagcagtctcacttctccaagccacgtgaacttgtctccaaatacagtcccagagttctcttactccagcagtgaagatgaattttatgatgctgatgaattccatcaaagtggctcatccccaaagcgcttaatagattcttctggatctgcctcagtcctgacacacagcagctcgggaaatagtctaaaacgcccagataccacagaatcacttaattcttccttgtccaatggaacaagtgatgctgacctgtttgattcacatgatgacagagatgatgatgcggaggcagggtctgtggaggagcacaagagcgttatcatgcatctcttgtcgcaggttagacttggaatggatcttactaaggtagttcttccaacgtttattcttgaaagaagatctcttttagaaatgtatgcagacttttttgcacatccggacctgtttgtgagcattagtgaccagaaggatcccaaggatcgaatggttcaggttgtgaaatggtacctctcagcctttcatgcgggaaggaaaggatcagttgccaaaaagccatacaatcccattttgggcgagatttttcagtgtcattggacattaccaaatgatactgaagagaacacagaactagtttcagaaggaccagttccctgggtttccaaaaacagtgtaacatttgtggctgagcaggtttcccatcatccacccatttcagccttttatgctgagtgttttaacaagaagatacaattcaatgctcatatctggaccaaatcaaaattccttgggatgtcaattggggtgcacaacatagggcagggctgtgtctcatgtctagactatgatgaacattacattctcacattccccaatggctatggaaggtctatcctcacagtgccctgggtggaattaggaggagaatgcaatattaattgttccaaaacaggctacagtgcaaatatcatcttccacactaaacccttctatgggggcaagaagcacagaattactgccgagattttttctccaaatgacaagaagtctttttgctcaattgaaggggaatggaatggtgtgatgtatgcaaaatatgcaacaggggaaaatacagtctttgtagataccaagaagttgcctataatcaagaagaaagtgaggaagttggaagatcagaacgagtatgaatcccgcagcctttggaaggatgtcactttcaacttaaaaatcagagacattgatgcagcaactgaagcaaagcacaggcttgaagaaagacaaagagcagaagcccgagaaaggaaggagaaggaaattcagtgggagacaaggttatttcatgaagatggagaatgctgggtttatgatgaaccattactgaaacgtcttggtgctgccaagcattaggttggaagatgcaaagtttatacctgatgatcagggcagtaggcataattcagcaacaaacaatcttcctttgggagaaacctgttcattccaatcttctaattacagtggttcctatctcagggatactggactttctgacgcagatgaacaattaaggggaaaagcttcccttttccctctgtggcagttacgattttgacttcagtcctgagaaaaacttcaggttttgaaaatcagatgatgtcttctccttttccaaacaccacacgttgaaagcatttataaatccaagtctgaaactctgcgctctagtactgctgttaagatacacaacttgtttcttagttcatataatctcgggatacacacacacacacacatatatatacacacacatacgtatacacacacatacatatatataaatatacctgatgccagatttttttcataaatattctgcctactgtaaatatgggttcctctgagttgttttagaaaattagcgcaatgtattaaaatcaagtgttaggaaatttcatggtcttacctacaataacttttattttggaattgaactattattaaattgtatctaatcctgg

SEQ ID NO: 8 - protein, 558 aa, Homosapien
  mouse
MVESIKHCIVLLQIAKSTINPVDAIYQPSPLEPVISTMPSQTVLPPEPVQLCKSEQRPSSLPVGPVLATLGHHQTPTPNSTGSGHSPPSSSLTSPSHVNLSPNTVPEFSYSSSEDEFYDADEFHQSGSSPKRLIDSSGSASVLTHSSSGNSLKRPDTTESLNSSLSNGTSDADLFDSHDDRDDDAEAGSVEEHKSVIMHLLSQVRLGMDLTKVVLPTFILERRSLLEMYADFFAHPDLFVSISDQKDPKDRMVQVVKWYLSAFHAGRKGSVAKKPYNPILGEIFQCHWTLPNDTEENTELVSEGPVPWVSKNSVTFVAEQVSHHPPISAFYAECFNKKIQFNAHIWTKSKFLGMSIGVHNIGQGCVSCLDYDEHYILTFPNGYGRSILTVPWVELGGECNINCSKTGYSANIIFHTKPFYGGKKHRITAEIFSPNDKKSFCSIEGEWNGVMYAKYATGENTVFVDTKKLPIIKKKVRKLEDQNEYESRSLWKDVTFNLKIRDIDAATEAKHRLEERQRAEARERKEKEIQWETRLFHEDGECWVYDEPLLKRLGAAKH

SEQ ID NO: 9 - DNA, 20 bp, Artificial Sequence
  Synthetic
tccacgatggacgccatctt

SEQ ID NO: 10 - DNA, 20 bp, Artificial Sequence
  Synthetic
gccagcaggagagccattcg

SEQ ID NO: 11 - DNA, 20 bp, Artificial Sequence
  Synthetic
aagatggcgtccatcgtgga

SEQ ID NO: 12 - DNA, 20 bp, Artificial Sequence
  Synthetic
cgaatggctctcctgctggc

SEQ ID NO: 13 - DNA, 21 bp, Artificial Sequence
  Synthetic
ccaagctctaatacgactcac

SEQ ID NO: 14 - DNA, 20 bp, Artificial Sequence
  Synthetic
ctcagccacaaatgttacac

SEQ ID NO: 15 - DNA, 32 bp, Artificial Sequence
  Synthetic
ctagctagcaccatggcgtccatcgtggaagg

SEQ ID NO: 16 - DNA, 35 bp, Artificial Sequence
  Synthetic
ataagaatgcggccgcgaggagaaaagggaagcgg

SEQ ID NO: 17 - DNA, 35 bp, Artificial Sequence
  Synthetic
ctagctagcaccatggctctcctgctggccgcttg

SEQ ID NO: 18 - DNA, 29 bp, Artificial Sequence
  Synthetic
cccgggcatgcttcacagcaccaagacgc